(19) United States
(12) Patent Application Publication
(10) Pub. No.: US 2006/0287607 A1
(43) Pub. Date: Dec. 21, 2006

(54) Multi-dimensional dynamical analysis

(75) Inventors: James Chris Sackellares, Gainesville, FL (US); Deng-Shan Shiau, Gainesville, FL (US); Linda K. Dance, Gainesville, FL (US); Leonidas D. Iasemidis, Scottsdale, AZ (US); Panos M. Pardalos, Gainesville, FL (US); Wanpracha A. Chaovalitwongse, Hillsborough, NJ (US)

Correspondence Address: BUCHANAN, INGERSOLL & ROONEY PC, POST OFFICE BOX 1404, ALEXANDRIA, VA, 22313-1404, US

(73) Assignees: University of Florida Research Foundation, Inc.; Arizona Board of Regents

(21) Appl. No.: 11/339,606

(22) Filed: Jan. 26, 2006

Related U.S. Application Data
(63) Continuation-in-part of application No. 10/673,329, filed on Sep. 30, 2003.
(63) Continuation-in-part of application No. 10/648,354, filed on Aug. 27, 2003.
(60) Provisional application No. 60/647,380, filed on Jan. 26, 2005.
(60) Provisional application No. 60/414,364, filed on Sep. 30, 2002.
(60) Provisional application No. 60/414,364, filed on Sep. 30, 2002.
(60) Provisional application No. 60/406,063, filed on Aug. 27, 2002.

(51) Int. Cl. A61B 5/04

(52) U.S. Cl. 600/544

(57) ABSTRACT

Techniques for investigating dynamical behavior of complex systems for monitoring, diagnosing, and predicting future behavior and trends are presented. A method includes generating a multi-dimensional representation for each signal acquired from corresponding channels of a multi-dimensional system, and generating dynamical profiles for each channel in accordance with one of multiple dynamical metrics. The method includes calculating multiple first statistical measures for a group of the channels that reflect a level of interaction among the channel group associated with at least one of the metrics. The method includes calculating in an initialization period a second statistical measure for each of the first statistical measures that reflect the association of the first statistical measures with related occurrences under investigation. The method includes selecting in the initialization period at least one of the metrics based on the second statistical measures, and identifying the first statistical measures corresponding to the selected metrics to characterize dynamical behavior.

### CROSS-REFERENCE TO RELATED APPLICATIONS

This application claims the benefit of U.S. Provisional Patent Application No. 60/647,380, filed Jan. 26, 2005. This application is a continuation-in-part of U.S. patent application Ser. No. 10/673,329, filed Sep. 30, 2003, which claims the benefit of U.S. Provisional Patent Application No. 60/414,364, filed Sep. 30, 2002. This application is also a continuation-in-part of U.S. patent application Ser. No. 10/684,354, filed Aug. 27, 2003, which claims the benefit of U.S. Provisional Patent Application No. 60/414,364, filed Sep. 30, 2002 and U.S. Provisional Patent Application No. 60/406,063, filed Aug. 27, 2002. U.S. Provisional Patent Application No. 60/647,380, U.S. patent application Ser. No. 10/673,329, and U.S. patent application Ser. No. 10/684,354 are all incorporated by reference herein in their entireties. This application is related to U.S. Pat. No. 6,304,775, issued Oct. 16, 2001, which is also incorporated by reference herein in its entirety.

### STATEMENT REGARDING FEDERALLY SPONSORED RESEARCH OR DEVELOPMENT

The research and development effort associated with the subject matter of this patent application was supported by the Department of Veterans Affairs and by the National Institute of Biomedical Imaging and Bioengineering of the National Institutes of Health (NIBIB/NIH) under grant no. R01EB002089.

### FIELD OF THE INVENTION

The present invention involves the field of signal processing. More particularly, the present invention involves multi-parameter processing of multi-channel time series signals associated with multi-dimensional systems.

### DESCRIPTION OF THE RELATED ART

Related U.S. Pat. No. 6,304,775 describes systems and methods capable of effectively generating true seizure warnings and predictions well in advance of impending seizures. The systems and methods described in U.S. Pat. No. 6,304,775 patent take advantage of the spatio-temporal characteristics exhibited by certain sites within the brain, when compared with the spatio-temporal characteristics exhibited by other sites within the brain, as these characteristics are noticeably different prior to an impending seizure as compared to the spatio-temporal characteristics exhibited by these same sites during seizure free intervals. In fact, these spatio-temporal characteristics may be noticeable hours, and in some cases, days before the occurrence of a seizure. As such, the systems and methods described in U.S. Pat. No. 6,304,775 use these differences as a seizure transition indicator.

### BACKGROUND OF THE INVENTION

Many areas of science (e.g., mathematics, physics, chemistry, biology, and medicine) have begun to investigate the dynamics of complex systems, such as multi-dimensional systems. A multi-dimensional system exhibits behavior autonomously or as a function of multiple variables in response to a system input. Example multi-dimensional systems include the nervous system (e.g., the brain), turbulent flow of fluids, certain complex chemical reactions, gene-environmental interactions, market behavior and other economic data, changes in the population of various species, and ecological behaviors, among others.

Mathematical approaches that stem from the theory of nonlinear dynamics and chaos theory have been used to investigate the dynamics of such complex systems. In addition, data mining approaches, such as global optimization, can be used to search for patterns present in massive amounts of data. Limitations in computational speed and data storage capacity of presently available computational systems, however, prevent these mathematical approaches from being used in combination as a tool for investigating complex systems.

What is needed, therefore, are techniques for investigating complex systems that can be used for research, monitoring and diagnosis, and for quickly understanding and predicting future behavior of the complex systems.

### BRIEF SUMMARY OF THE INVENTION

Techniques are provided herein for investigating dynamical behavior and underlying mechanisms of data measurements from complex systems for purposes of monitoring, diagnosing, and predicting future behavior and trends.

In accordance with a first aspect of the present invention, a method of monitoring dynamical behavior of a multi-dimensional system includes the following steps. First, a multi-dimensional representation is generated for each signal acquired from corresponding channels of the multi-dimensional system. Dynamical profiles are then generated for each channel based on the corresponding multi-dimensional representations. Each dynamical profile reflects dynamical characteristics of the associated channel in accordance with one of multiple dynamical metrics. Next, multiple first statistical measures are calculated for each of at least one group of two or more of the channels. Each first statistical measure reflects a level of interaction among the channel group associated with at least one of the dynamical metrics.

Next, in an initialization period, a second statistical measure for each of the first statistical measures is calculated. The second statistical measure reflects the association of the first statistical measure with the related occurrences (i.e., events and/or transitions, such as seizure-related events and/or transitions) under investigation. Additionally, in the initialization period, at least one of the dynamical metrics is selected based on the calculated second statistical measures, and the first statistical measures corresponding to the selected dynamical metrics are identified to characterize the dynamical behavior of the multi-dimensional system.

In accordance with a second aspect of the present invention, a computer readable medium has a program stored therein, which when executed causes a processor to perform the following functions for monitoring dynamical behavior of a multi-dimensional system. The program causes the processor to generate multi-dimensional representations for signals acquired from corresponding channels of the multi-dimensional system, and generate multiple dynamical profiles for each channel based on the corresponding multi-dimensional representations. Each dynamical profile reflects dynamical characteristics of the associated channel in accordance with one of multiple dynamical metrics. The program further causes the processor to calculate multiple first statistical measures for each of at least one group of two or more of the channels. Each first statistical measure reflects a level of interaction among the channel group associated with at least one of the dynamical metrics.

The program further causes the processor to calculate in an initialization period a second statistical measure for each of the first statistical measures generated. The second statistical measure reflects the association of the first statistical measure with the related occurrences (i.e., events and/or transitions, such as seizure-related events and/or transitions) under investigation. The program further causes the processor to select in the initialization period at least one of the dynamical metrics based on the calculated second statistical measures, and identify the first statistical measures corresponding to the selected dynamical metrics to characterize the dynamical behavior of the multi-dimensional system.

In accordance with a third aspect of the present invention, a system for monitoring dynamical behavior of a multi-dimensional includes a processing device that executes the following steps. The processing device executes generating a multi-dimensional representation for each signal acquired from corresponding channels of the multi-dimensional system, and generating multiple dynamical profiles for each channel based on the corresponding multi-dimensional representations. Each dynamical profile reflects dynamical characteristics of the associated channel in accordance with one of multiple dynamical metrics.

The processing device further executes calculating multiple first statistical measures for each of at least one group of two or more of the channels. Each first statistical measure reflects a level of interaction among the channel group associated with at least one of the dynamical metrics. The processing device further executes calculating in an initialization period a second statistical measure for each of the first statistical measures generated. The second statistical measure reflects the association of the first statistical measure with the related occurrences (i.e., transitions and/or events such as, seizure-related transitions and/or events) under investigation. The processing device further executes selecting in the initialization period at least one of the dynamical metrics based on the calculated second statistical measures, and identifying the first statistical measures corresponding to selected dynamical metrics to characterize the dynamical behavior of the multi-dimensional system.

### BRIEF DESCRIPTION OF THE DRAWING FIGURES

The objects and advantages of the present invention will be understood by reading the following detailed description in conjunction with the drawings in which:

FIGS. 1(a)-(e) illustrates an exemplary, single channel EEG signal acquired as a patient transitions through the various stages of an epileptic seizure;

FIG. 2 illustrates a typical, continuous multi-channel EEG segment prior to and during seizure onset;

FIG. 3 is a flowchart depicting techniques for providing early ISW, SSPD and TISP in accordance with exemplary embodiments of the present invention;

FIGS. 4A and 4B illustrate the placement and use of different electrodes and electrode configurations;

FIGS. 5A and 5B illustrate an EEG signal associated with a representative electrode channel over an epoch and the corresponding phase space portraits containing the attractor reconstructed from the EEG signal using the Method of Delays;

FIG. 6 depicts the process of generating chaoticity profiles associated with each of multiple chaoticity metrics, for one or more channels, and therefrom, generating statistical profiles for one or more channel pairs;

FIG. 7 illustrates the L_{MAX} profiles associated with each of a representative number of channel pairs;

FIG. 8 illustrates a procedure for comparing L_{MAX} profiles (e.g., estimations of T-index profiles) for the representative number of channel pairs shown in FIG. 7;

FIG. 9 illustrates the TISP feature in accordance with exemplary embodiments of the present invention;

FIG. 10 illustrates an on-line system that incorporates the ISW, SSPD and TISP features of the present invention;

FIG. 11 is a flowchart providing example steps for monitoring dynamical behavior of a multi-dimensional system in accordance with exemplary embodiments of the present invention; and

FIG. 12 illustrates an example system for monitoring dynamical behavior of a multi-dimensional system in accordance with exemplary embodiments of the present invention.

### DETAILED DESCRIPTION OF THE INVENTION

Overview

Techniques are provided herein for investigating dynamical behavior and underlying mechanisms of data measurements from complex deterministic or stochastic systems for purposes of monitoring, diagnosing, and predicting future behavior and trends. Research laboratories, health care systems, financial services, telecommunication companies, universities, research clinicians, governments, economists, among others, can all benefit from such techniques for analyzing complex systems.

In accordance with exemplary embodiments of the present invention, a general technique for analyzing data acquired from a complex system (or generated by some mathematical model) in order to discover spatiotemporal properties of the system dynamics is as follows. First, single to multiple channels (i.e., dimensions) of data are read for analysis. Then, data from each channel are broken into discrete time windows, referred to herein as “epochs,” and the dynamical characteristics of each epoch for each channel are described quantitatively in terms of quantitative descriptors. The quantitative descriptors for each epoch from each channel are calculated by embedding the data, initially represented as a one-dimensional time series, into a multidimensional state-space. The quantitative descriptors together can be translated into a multi-dimensional vector (or analyzed alone as a time series). Lastly, the mathematical descriptors of the dynamics among a channel group can be compared with some type of statistic (e.g., T-index) that provides an indication as to how they are interacting over time in order to obtain information about spatial as well as temporal dynamics of the system.

The techniques described herein for multi-dimensional dynamical analysis of complex systems can be used to analyze the natural behavior of complex systems (i.e., no interventions) or the results of experiments (e.g., interventions or perturbations of the system by the investigator). Both types of investigative approaches are commonly used in the analysis of complex systems by scientists and engineers. For example, these investigative approaches can be used to model the behavior of the normal and epileptic brain, and to model the response of the brain to interventions aimed at preventing or terminating epileptic seizures. However, similar approaches can also be used to analyze the properties of systems (e.g., system identification) for purposes of developing direct or model based controls.

The techniques described herein for multi-dimensional dynamical analysis of complex systems are advantageous for predicting events of complex systems with sufficient warning time to allow for intervention in a real time, on-line mode, and for understanding the mechanisms underlying the behavior of such complex systems.

Multi-Dimensional Dynamical Analysis Methods and Systems

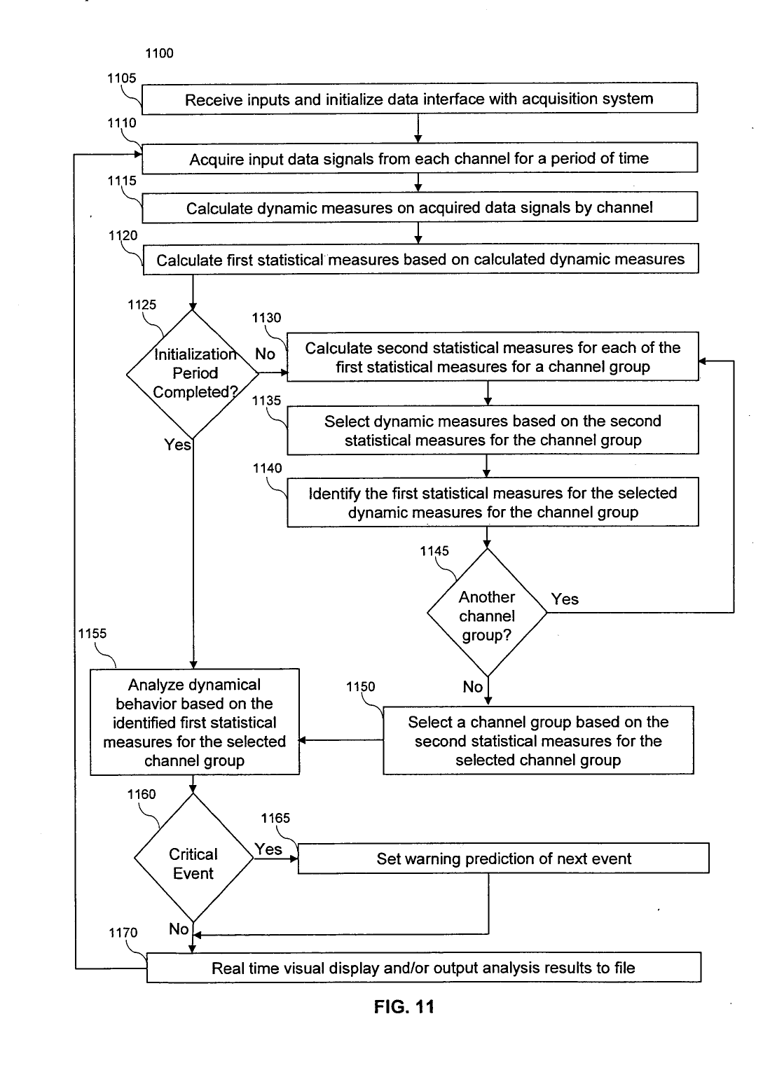

FIG. 11 is a flowchart providing example steps for a process 1100 for monitoring dynamical behavior of a multi-dimensional system in accordance with exemplary embodiments of the present invention. The steps of FIG. 11 do not necessarily have to occur in the order shown, as will be apparent to persons skilled in the relevant art(s) based on the teachings herein. Other operational and structural embodiments will be apparent to persons skilled in the relevant art(s) based on the following discussion. These steps are described in detail below.

It should be noted that in accordance with an embodiment of the present invention, the method illustrated in FIG. 11 is employed in conjunction with a computer-based system, where the method can be implemented in hardware, software, firmware, or combinations thereof.

In step 1105, inputs, such as a selection of measures and parameters for calculations, are received, and a data interface with an acquisition system of the multi-dimensional system is initialized. In an embodiment, a selection of multiple dynamical metrics and/or parameters is received for quantifying the dynamical properties of the spatio-temporal responses of each channel of the multi-dimensional system. Such dynamical metrics include, for example, STL_{MAX} (rate of divergence), Ω_{MAX} (rate of change in angular frequency), ApEn (approximate entropy or regularity statistic), PM-ApEn (pattern-match approximate entropy), h_{μ} (Pesin's Identity) and d_{L} (Lyapunov Dimension). In another embodiment, a selection of a statistical test for determining a level of interaction between the channels that make up one or more channel groups is received. The statistical test includes, for example, a nonparametric randomized block ANOVA (Analysis of Variance) test or a T-statistic test.

In step 1110, input data signals from each channel of the multi-dimensional system are acquired for a period of time. For example, data signals for processing can be acquired within overlapping or non-overlapping “sliding” time windows.

In step 1115, dynamic measures on the acquired data signals are calculated by channel based on the selection of measures received in step 1105. The dynamic measures generated quantify the dynamical properties of the spatio-temporal responses of each channel of the multi-dimensional system.

In step 1120, first statistical measures based on the dynamic measures calculations performed in step 1115 are calculated for at least one group of two or more of the channels. The selected statistical test received in step 1105 can be used. For example, the nonparameteric randomized block ANOVA test can be applied to generate a sequence of X²-index values. The first statistical measures generated can be used to assess a level of interaction, such as correlation, among the channel group.

In step 1125, it is determined if an initialization period has been completed. During the initialization period, parameters are initialized for each channel group according to the behavior of the system in response to transitions and/or events under investigation (e.g., seizure-related transitions and/or events). If the initialization period has not yet been completed, then, in step 1130, second statistical measures are calculated for each of the first statistical measures calculated in step 1120 for a particular channel group. The second statistical measures reflect the association of the first statistical measures with the related transitions and/or events that occur during the initialization period. For example, the second statistical measures might quantify a level of accuracy with which the first statistical measures predict the related transitions and/or events.

In step 1135, at least one dynamic measure is selected based on the calculated second statistical measures for the channel group. In step 1140, the first statistical measures corresponding to the selected dynamic measures for the channel group are identified. For example, the most robust first statistical measures, such as the first statistical measures that exhibit a desired level sensitivity and specificity during the initialization period, might be identified.

In step 1145, it is determined if any additional channel groups have to be analyzed during the initialization period. If additional channel groups have not yet been analyzed, then process 1100 repeats steps 1130, 1135, and 1140 for each additional channel group. If all of the channel groups have been analyzed, then, in step 1150, at least one channel group is selected based on the second statistical measures calculated for the respective channel groups. As described above for the first statistical measures identified in step 1140, the most robust channel group or groups during the initialization period might be selected. After step 1150, process 1100 proceeds to step 1155.

If, in step 1125, it is determined that the initialization period has been completed, then, in step 1155, dynamical behavior of the multi-channel system is analyzed based on the identified first statistical measures for the channel group or groups selected in step 1150. In step 1160, it is determined if a critical event (e.g., a condition indicative of an impending seizure) has occurred. If it is determined that a critical event has occurred, then, in step 1165, a warning of a prediction of a next event is set and process 1100 proceeds to step 1170. If it is determined that a critical event has not occurred, then process 1100 proceeds directly to step 1170.

Optionally, in step 1170, the analysis results can be displayed in real time and/or output to a file. For example, the input data signals acquired in step 1110, the dynamic measures calculated in step 1115, and the statistical measures calculated in steps 1120 and 1130 can be displayed in real time and/or output to a file. After step 1170, process 1100 returns to step 1110 to continue acquiring input signals.

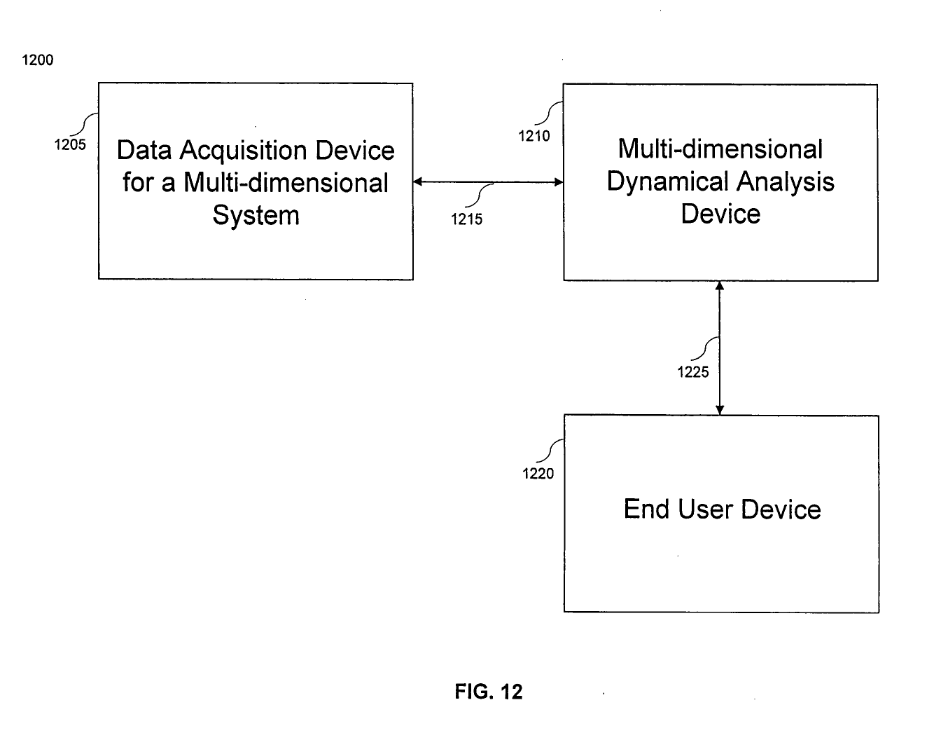

FIG. 12 illustrates an example system 1200 for monitoring dynamical behavior of a multi-dimensional system in accordance with exemplary embodiments of the present invention. For example, system 1200 can be used to implement process 1100, described above, in conjunction with FIG. 11.

System 1200 includes a data acquisition device for a multi-dimensional system 1205, a multi-dimensional dynamical analysis device 1210, and an optional end user workstation 1220. Data acquisition device 1205 is coupled to multi-dimensional dynamical analysis device 1210 by a communications medium (e.g., a wired or wireless communications line) 1215. Similarly, multi-dimensional dynamical analysis device 1210 is coupled to end user device 1220 by a communications medium (e.g., a wired or wireless communications line) 1225.

Data acquisition device 1205 monitors incoming signals from one or more sensors of the multi-dimensional system. In the example of EEG analysis, data acquisition device 1205 can be implemented with a Nicolet BMSI™ 6000 Long-Term Monitoring System, a Viasys NicOne Long-Term Monitoring System, a Compumedics Long-Term Monitoring System, an XLTEK EEG Long-Term Monitoring System, a Stellate Harmonie EEG Acquisition Workstation, and the like.

Multi-dimensional dynamical analysis device 1210 reads the acquired signals from data acquisition device 1205 over communications medium 1215. Multi-dimensional dynamical analysis device 1210 continuously calculates various signal properties such as signal energy, approximate entropy (ApEn), dynamical phase, maximum short-term Lyapunov exponent (STL_{MAX}), among other properties, for each channel through analysis of sequential overlapping or non-overlapping time windows of variable length. Multi-dimensional dynamical analysis device 1210 also makes ongoing comparisons of signal properties such as information flow, mutual information, coherence, cross-ApEn, and statistical and dynamical interactions between the acquired signals over time.

By implementing the techniques for monitoring dynamical behavior described herein, multi-dimensional dynamical analysis device 1210 can detect temporal, spatial, or spatiotemporal patterns and trends through visual display of the signal, signal properties, and interactions described above. Multi-dimensional dynamical analysis device 1210 can also detect states, state transitions, and self-organizing patterns. Furthermore, multi-dimensional dynamical analysis device 1210 can detect evidence for deterministic (linear or nonlinear) and/or stochastic processes and can be used to predict short and long term trends and state transitions.

In an embodiment, multi-dimensional dynamical analysis device 1210 includes an analysis workstation capable of real time visualization of the acquired signals, the dynamic measure calculations, and the statistical analysis with real time event prediction and event detection. Analysis results can be displayed graphically or numerically for purposes of long term monitoring, diagnostics, and prediction.

Optionally, multi-dimensional dynamical analysis device 1210 communicates analysis results to end user device 1220 over communications medium 1225. In this way, real time event prediction and event detection, in addition to event intervention, can be communicated to end user 1220.

One application of the multi-dimensional dynamical analysis methods and systems described above is investigating multi-channel EEG's of patients with epilepsy for seizure prediction. More particularly, U.S. Pat. No. 6,304,775 describes, among other things, a technique that provides timely impending seizure warning (ISW), seizure susceptibility period detection (SSPD) and time to impending seizure prediction (TISP). The technique involves acquiring electrical or electromagnetic signals generated by the brain, where each signal corresponds to a single EEG electrode or channel. Each signal is pre-processed (e.g., amplified, filtered, digitized) and sampled. This results in a sequence of digital samples for each signal over a period of time, referred to therein as an epoch. The samples are then used to generate a phase-space portrait for each signal epoch.

For each phase-space portrait, a parameter reflecting rate of divergence is computed based on adjacent trajectories in the phase space, where rate of divergence, in turn, is one parameter that reflects the chaoticity level of the corresponding signal. In U.S. Pat. No. 6,304,775, the parameter used is the short-term, largest Lyapunov exponent (STL_{MAX}).

In general, the STL_{MAX} values associated with each EEG signal (i.e., each EEG channel) are compared to the STL_{MAX} values associated with each of the other channels. The comparisons are preferably achieved by applying a T-statistic, which results in a sequence of statistical values, or T-index values, for each channel pair, where a sequence of T-index values represents a level of correlation or entrainment between the spatio-temporal response associated with the two channels that make up each channel pair.

The technique, when first employed, goes through an initialization period. During this initialization period, a number of “critical” channel pairs is identified, where U.S. Pat. No. 6,304,775 generally defines a critical channel pair as a pair of channels that exhibits a relatively high level of entrainment (i.e., relatively low T-index values for a predefined period of time) prior to seizure onset.

During the initialization period, a patient may experience one or more seizures. After each, the list of critical channel pairs is updated. Eventually, the list of critical channel pairs is considered sufficiently refined, and the initialization period is terminated. Thereafter, the ISW, SSPD and TISP functions may be activated and the T-index values associated with the critical channel pairs are monitored and employed in generating timely ISWs, SSPDs and/or TISPs.

Co-pending U.S. patent application Ser. No. 10/648,354 describes both methods and systems that optimize the critical channel selection process. Optimization is achieved in several ways. First, the selection is achieved more efficiently as it is based on a limited amount of statistical data (e.g., T-index data) within a pre-defined time window preceding, and in some instances, following seizure-related events. Critical channel selection is further optimized by selecting and reselecting critical channels for each of a number of predictors, where a predictor is a given number of critical channel groups “x”, a given number of channels per group “y”, and a given total number of channels “N.”

Additionally, co-pending U.S. patent application Ser. No. 10/673,329 focuses on generating signals that more effectively reflect the non-linear dynamical characteristics of a multi-dimensional system, such as the brain. In U.S. Pat. No. 6,304,775, chaoticity profiles based on, for example, STL_{MAX}, are generated from corresponding time-series signals, where each profile reflects the chaoticity or rate of divergence of a corresponding channel. However, other metrics may also provide a good basis for measuring the dynamical characteristics of a system such as the brain. Thus, multiple dynamical metrics are considered.

While investigating multi-channel EEG's of patients with epilepsy for seizure prediction is one important application of the multi-dimensional dynamical analysis methods and systems described above in conjunction with FIGS. 11 and 12, these methods and systems can also be implemented more generally for use by scientists, economists, and meteorologists, among others. For example, other applications include other areas of health care, including the function of normal biological systems, such as the nervous system, heart, perception, memory, functions, homeostasis, reaction to environmental stimuli, sleep-wake cycles, cardiac arrhythmia, respiratory cycles and motor function; and disorders of biological systems, such as coma, stroke, movement disorders, cognitive disorders, heart disease, sleep disorders, head injury, learning disabilities, attention deficit disorder, dyslexia, clinical depression and migraine headaches.

The methods and systems described above in conjunction with FIGS. 11 and 12 can also be used to investigate, monitor, and diagnose physical phenomena, such as weather, geological phenomena, chemical reactions, mechanical systems, electronic systems, telecommunication systems, and financial trends (e.g., market behavior), to name a few. For example, in the case of a weather system, seismic data signals from sensors located in a particular geographic area can be analyzed to predict earthquakes, and, in the case of a financial system, data pertaining to a portfolio of stocks can be analyzed to predict market events. A detailed description of an exemplary embodiment for investigating multi-channel EEG's of patients with epilepsy for seizure prediction follows.

### Detailed Description of an Exemplary Embodiment

The brain is an example of a multi-dimensional system that also exhibits chaotic behavior during normal operation. However, in a relatively significant percentage of the human population, the brain experiences deterministic, abnormal episodes characterized by less chaotic behavior. This abnormal behavior may be caused by a wide variety of conditions. Epilepsy is one of these conditions.

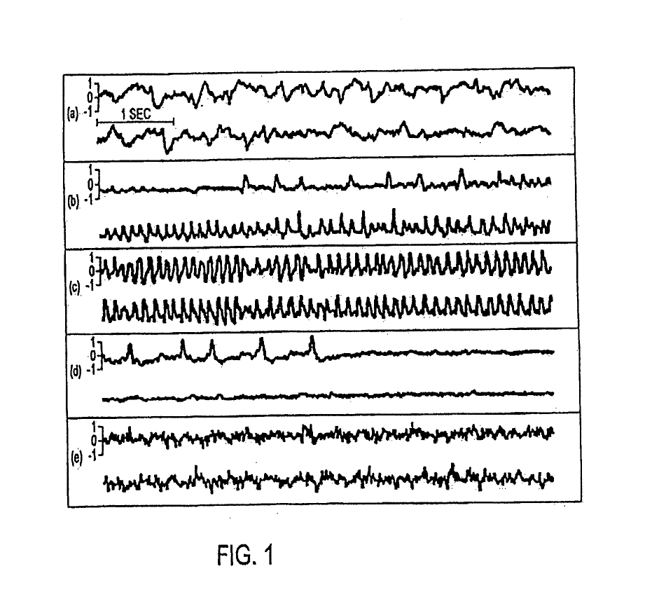

Seizures, including epileptic seizures, are multiple stage events. The various stages include a preictal stage, an ictal stage, a postictal stage and an interictal stage. FIGS. 1(a-e) illustrate an exemplary electroencephalogram (EEG) signal, recorded from an electrode overlying an epileptogenic focus, as a patient transitions through the various stages of an epileptic seizure. More specifically, FIG. 1(a) illustrates a time sequence of the EEG signal during the preictal stage, which represents the period of time preceding seizure onset. FIG. 1(b) illustrates a time sequence of the EEG signal during the transition period between the preictal stage and the ictal stage, which include seizure onset. It follows that FIG. 1(c) then reflects the EEG signal during the ictal stage that is within the epileptic seizure, where the ictal stage begins at seizure onset and lasts until the seizure ends. FIG. 1(d), like FIG. 1(b), covers a transitional period. In this case, FIG. 1(d) illustrates a time sequence of the EEG signal during the transition from the ictal stage to the postictal stage, and includes the seizure's end. FIG. 1(e) then illustrates the EEG signal during the postictal stage, where the postictal stage covers the time period immediately following the end of the seizure.

As stated, the preictal stage represents a period of time preceding seizure onset. More importantly, the preictal stage represents a time period during which the brain undergoes a dynamic transition from a state of spatio-temporal chaos to a state of spatial order and reduced temporal chaos. Although it will be explained in greater detail below, this dynamic transition during the preictal stage is characterized by the dynamic entrainment of the spatio-temporal responses associated with various cortical sites. As set forth in U.S. Pat. No. 6,304,775 and co-pending U.S. patent application Ser. No. 10/648,354, the dynamic entrainment of the spatio-temporal responses can be further characterized by: (1) the progressive convergence (i.e., entrainment) of the maximum Lyapunov exponent values (i.e., L_{MAX}) corresponding to each of the various, aforementioned cortical sites, where L_{MAX} provides a measure of chaoticity associated with the spatio-temporal response of a corresponding cortical site; and (2) the progressive phase locking (i.e., phase entrainment) of the L_{MAX} profiles associated with the various cortical sites.

The dynamic entrainment of the spatio-temporal responses may also be characterized by the convergence and/or phase-locking of profiles generated based on dynamical metrics other then L_{MAX}. Thus, in an embodiment of the present invention, other metrics (i.e., other dynamical metrics) are taken into consideration, and they include the rate of change in angular frequency Ω_{MAX}, approximate entropy ApEn, Pattern-match ApEn, Pesin's Identity h_{μ}, and the Lyapunov Dimension d_{L}. Each of these chaoticity metrics will be described in greater detail below.

As one skilled in the art will readily appreciate, an EEG signal, such as any of the EEG signals depicted in FIGS. 1 (a-e), is a time series signal that represents a temporal response associated with the spatio-temporal interactions of a particular portion of the brain where the corresponding electrode happens to be located. Since, the brain is a complex, multi-dimensional system, EEG signals and other known equivalents do not and cannot visibly reflect the true spatio-temporal characteristics exhibited by the brain. Thus, traditional linear and nonlinear methods of processing EEG signals for the purpose of providing seizure prediction and/or warning have proven to be generally ineffective as the critical spatio-temporal characteristics exhibited by the brain during the preictal stage cannot be detected from EEG signals alone. Yet, these critical spatio-temporal characteristics exist long before seizure onset, in some cases, days before seizure onset. As such, these spatio-temporal characteristics exhibited by the brain during the preictal stage are essential to any true seizure prediction scheme.

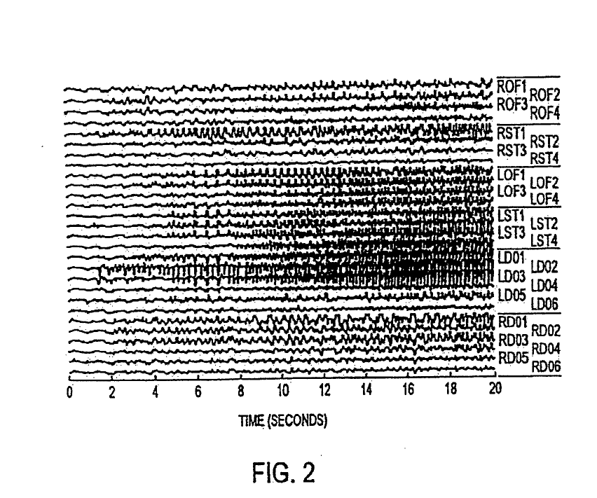

To better illustrate the deficiency of EEG signals, FIG. 2 shows a 20 second EEG segment covering the onset of a left temporal lobe seizure. The EEG segment of FIG. 2 was recorded from 12 bilaterally placed hippocampal depth electrodes (i.e., electrodes LTD1-LTD6 and RTD1-RTD6), 8 subdural temporal electrodes (i.e., electrodes RST1-RST4 and LST1-LST4), and 8 subdural orbitofrontal electrodes (i.e., electrodes ROF1-ROF4 and LOF1-LOF4). Seizure onset begins approximately 1.5 seconds into the EEG segment as a series of high amplitude, sharp and slow wave complexes in the left depth electrodes, particularly in LTD1-LTD3, though most prominently in LTD2. Within a matter of seconds, the seizure spreads to right subdural temporal electrode RST1, and then to the right depth electrodes RTD1-RTD3. Of particular importance is the fact that the EEG signals appear normal prior to seizure onset approximately 1.5 seconds into the EEG segment.

The present invention provides an early ISW based on the aforementioned spatio-temporal changes that occur during the preictal stage. The present invention provides this capability even though EEG signals do not manifest any indication of an impending seizure during the preictal stage, as illustrated in FIG. 2. In addition to providing an ISW, the present invention is also capable of providing a seizure susceptibility period detection (SSPD), that is, the presence of abnormal brain activity long before the occurrence of a seizure, for example, during an interictal period days before a seizure. Furthermore, the present invention is capable of providing a time to impending seizure prediction (TISP), wherein the TISP reflects an amount of time that is expected to elapse before seizure onset.

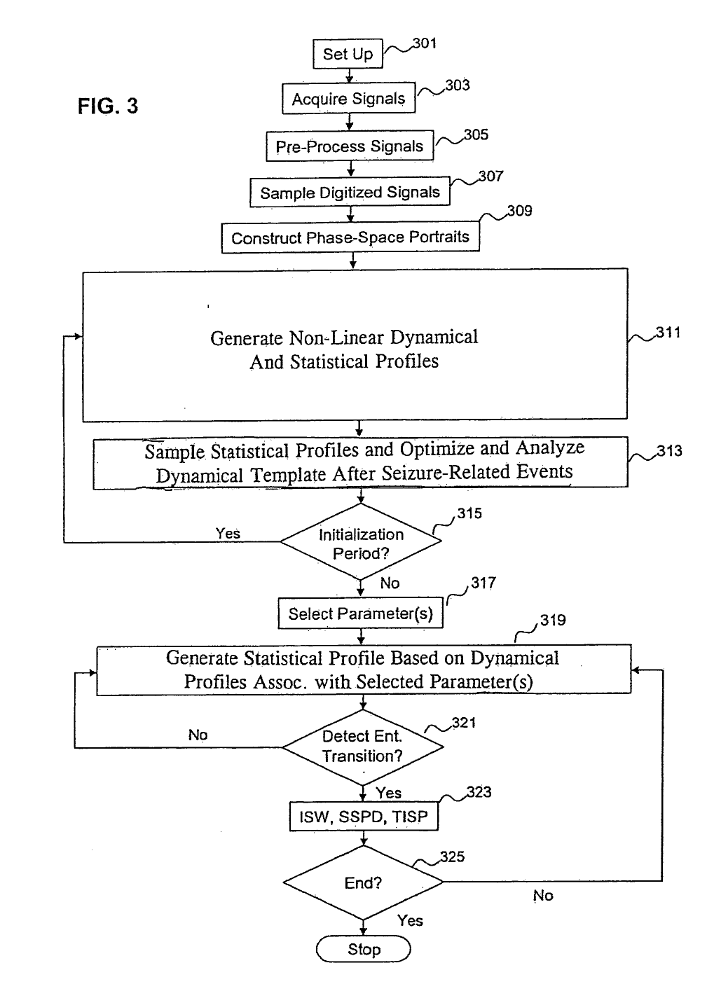

FIG. 3 is a flowchart depicting an exemplary embodiment of the present invention. In general, the method illustrated by the flowchart of FIG. 3 involves selecting one dynamical metrics or a combination of dynamical metrics from amongst multiple dynamical metrics at the end of an initialization period. Thereafter, an ISW, SSPD and/or TISP are issued based on a statistical measure associated with at least one channel group.

It should be noted that in accordance with an embodiment of the present invention, the methods illustrated in FIGS. 3A and 3B are employed in conjunction with a computer-based system, where the methods are implemented in software, firmware or a combination of both. For ease of discussion, the methods are generally referred to herein as the “algorithm.”

Turning now to the individual steps associated with the method of FIG. 3, step 301 represents a setup procedure. During the setup procedure, the operator of the computer-based system, such as a physician or clinician, selects or defines various metrics, parameter values and threshold levels used by the algorithm during execution. For example, it is at this point that the operator may be given the option of selecting, from a list of possible choices, the multiple dynamical metrics that are to be used by the algorithm to ultimately determine the behavior of the system under test (e.g., the brain). Similarly, the operator may be given the option of selecting the statistical test that the algorithm will use to determine the correlation level of the channels that make up one or more channel groups. In an embodiment, the statistical test that is used is the nonparametric randomized block ANOVA (Analysis of Variance) test, or X²-statistic, where the values produced as a result of applying an X²-statistic are referred to herein as X²-index values.

Still further, the operator may, during setup step 301, define the value of various thresholds. One such threshold is referred to herein as the disentrainment threshold X²_{D}, where X²_{D} represents a X²-index value threshold, and where a corresponding channel group is considered significantly and statistically disentrained when the X²-index profile associated therewith exceeds this threshold. Another threshold is referred to herein as the entrainment threshold X²_{E}, where X²_{E} represents a X²-index value threshold, below which a corresponding channel group is considered sufficiently and statistically entrained.

In accordance with step 303, the algorithm begins acquiring electrical or electromagnetic signals generated by the brain. In an embodiment, each signal corresponds with a single EEG channel. Each signal is pre-processed, per step 305, where pre-processing typically includes signal amplification, filtering and digitization. Each digitized signal is then sampled, as shown in step 307, and based on each sampled signal, a corresponding sequence of phase-space portraits are generated from each signal, as shown by step 309, where each phase-space portrait is based on samples falling within in a corresponding time window called an epoch.

In step 311, the algorithm first generates a number of dynamical data sequences or dynamical profiles for each channel. The dynamical profiles associated with a given channel are generated as a function of a phase-space portrait associated with that channel, where each of the dynamical profiles reflects the non-linear dynamical characteristics of the spatio-temporal response for that channel in accordance with a corresponding one of the multiple dynamical metrics. In FIG. 3, six exemplary dynamical metrics are shown: STL_{MAX}, Ω_{MAX}, ApEn, PM-ApEn, h_{μ} and d_{L}. However, it will be understood that dynamical metrics other than or in addition to those shown in FIG. 3 may be employed.

In an embodiment of the present invention, each of the dynamical profiles generated during step 311, for a given channel, may actually reflect an average dynamical measurement, where the average dynamical measurement may be derived, for example, by averaging a sequence of dynamical data values falling within overlapping or non-overlapping “sliding” time windows. Further in accordance with an embodiment of the present invention, the dynamical profiles associated with each dynamical metrics, for each channel, are generated by the algorithm using parallel processing techniques that are well known to those of skill in art.

Further in accordance with step 311, the algorithm generates several statistical measures for each of at least one channel group. As stated previously, the algorithm may do so by applying a nonparameteric randomized block ANOVA test. For a given channel group, the statistical measures are a function of the dynamical profiles, associated with each of the channels that make up the channel group, corresponding to one or a combination of the multiple dynamical metrics.

It will be understood that the nonparameteric randomized block ANOVA test is exemplary, where the statistical profiles generated as a result of applying this test comprise a sequence of X²-index values, and where each sequence of X²-index values are referred to herein as an X²-index profile. Accordingly, the algorithm may be set up so that it applies other statistical tests for testing the difference among channels with multiple parameter inputs.

In step 313, a limited sample of values (e.g., a sample falling within one or more predefined time windows) from each X²-index profile associated with at least one channel group is obtained following each of several seizure-related events. A seizure-related event may be an actual seizure or what is called an entrainment transition (i.e., an event characterized by a relatively high level of correlation amongst one or more channel pairs as indicated by relatively low X²-index values). The time window may, for example, be a 10 or 20-minute time window preceding or mostly preceding the seizure-related event.

A seizure may be detected using any of a number of techniques. For example, a seizure may be detected by an attending clinician, who does so by physically observing the behavior of the patient. Alternatively, a seizure may be detected by the algorithm itself, for example, by detecting a rapid decrease in the values associated with one or more X²-index profiles, followed by a rapid increase in the one or more X²-index profiles. As stated in U.S. Pat. No. 6,304,775, a seizure may also be detected by observing certain EEG signal manifestations indicative of a seizure. Other methods of seizure detection may be employed.

If a seizure is detected, the algorithm will, in accordance with an embodiment of the present invention, mark the occurrence of the seizure, for example, by setting a status flag and storing the time associated with seizure onset in memory. The algorithm may set the status flag and store seizure onset time automatically after it detects the seizure or in response to an action taken by the clinician. It is the setting of the status flag that causes the algorithm to sample the several X²-index profiles generated for each of the at least one channel groups, as indicated in step 313.

An entrainment transition event, on the other hand, may be detected if and when the algorithm determines that the value associated with one or more X²-index profiles has dropped below a predefined entrainment threshold X²E. In an alternative embodiment, the X²-index profiles would have to first exceed the X²_{D}, prior to falling below the entrainment threshold X²_{E} to constitute an entrainment transition event.

When the method illustrated in FIG. 3 is first employed, for instance, when it is used in conjunction with a new patient, there is an initialization period. The primary purpose of the initialization period is to provide a period of time during which the algorithm optimizes a dynamical template, where the dynamical template reflects each of the several X²-index profiles generated for the at least one channel group, as sampled over a number of seizure-related events during the transition period, as indicated by step 313, and the “YES” path out of decision step 315. More specifically, the dynamical template provides an indication and/or quantification of the sensitivity level associated with each X²-index profile with respect to impending seizure prediction during the various transitional states (e.g., interictal, preictal, ictal and post-ictal states). As one skilled in the art will readily appreciate, the dynamical template may quantify the sensitivity level in terms of a number of true seizure predictions per a total number of seizure-related events during the initialization period and/or a number of false predictions per a total number of seizure-related events. Moreover, one skilled in the art will understand that those X²-index profiles that exhibit a relatively high number of true predictions versus false predictions are likely to exhibit relatively low X²-index values just prior to or during the seizure-related events.

At the end of the initialization period, as indicated by the “NO” path out of decision step 315, and step 317, the algorithm selects one of or a combination of dynamical metrics and parameters, from amongst the other dynamical metrics and parameters, where the selected one or combination of dynamical metrics corresponds with the one X²-index profile that, during the initialization period, exhibited the best sensitivity level (i.e., provided the best indication of impending seizures) compared to the other X²-index profiles generated for the at least one channel group.

After the initialization period and the selection of one or a combination of dynamical metrics, it will be understood that the algorithm continues to acquire, pre-process, digitize and construct phase-space portraits as shown in steps 303 through 309. Per step 319, the algorithm then begins generating dynamical profiles based on the selected at least one or combination of dynamical metrics, for each channel. From these dynamical profiles, the algorithm generates a corresponding X²-index profile for at least one channel group.

In addition to generating an X²-index profile for each of at least one channel group, based on corresponding dynamical profiles associated with the selected one or combination of dynamical metrics, the algorithm monitors each X²-index profile. In accordance with decision step 321, the algorithm determines whether the conditions are indicative of an entrainment transition. The algorithm may do so, as explained above, by determining whether the X²-index values associated with the X²-index profile for one or more channel groups have dropped below the entrainment threshold X²_{E}, or alternatively, exceeded the disentrainment threshold X²_{D} prior to dropping below the entrainment threshold X²_{E}. If the algorithm determines the conditions do not indicate an entrainment transition, in accordance with the “NO” path out of decision step 321, the algorithm continues to generate the dynamical profiles based on the selected one or combination of dynamical metrics, and it continues to generate a corresponding X²-index profile for each of at least one channel group. If, however, the algorithm determines the conditions reflect an entrainment transition, in accordance with the “YES” path out of decision step 321, the algorithm will issue an ISW, SSPD and/or TISP, per step 323. A more detailed discussion of the ISW, SSPD and TISP is provided below.

Certain steps associated with the method illustrated in FIG. 3 will now be described in greater detail. For example, step 303 involves acquiring electrical or electromagnetic signals generated by the brain. In accordance with an embodiment of the present invention, electrodes are used to record electrical potentials, where each electrode corresponds to a separate channel, and where recordings are made using differential amplifiers. In referential recordings, one of the electrodes is common to all channels. The electrodes are strategically placed so that the signal associated with each channel is derived from a particular anatomical site in the brain. Electrode placement may include, for example, surface locations, wherein an electrode is placed directly on a patient's scalp. Alternatively, subdural electrode arrays and/or depth electrodes are sometimes employed when it is necessary to obtain signals from intracranial locations. One skilled in the art will appreciate; however, that the specific placement of the electrodes will depend upon the patient, as well as the application for which the signals are being recorded.

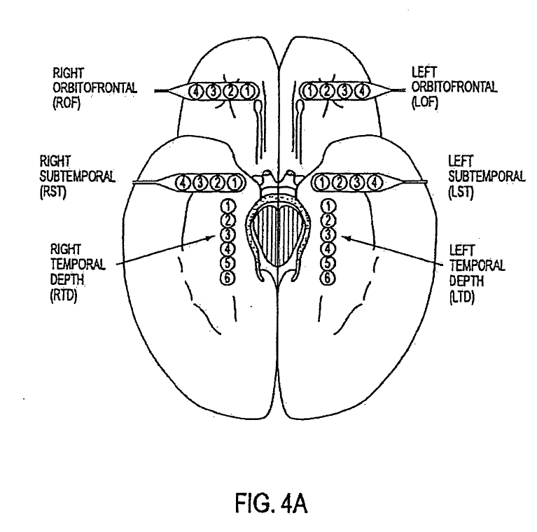

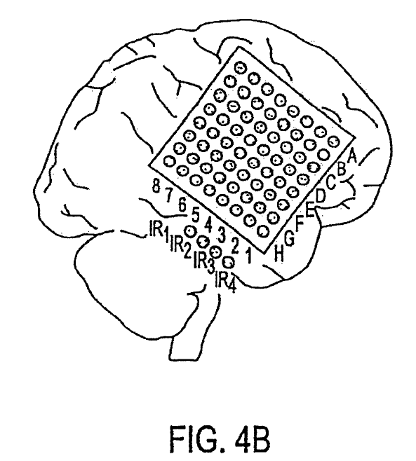

FIG. 4A provides a view from the inferior aspect of the brain and exemplary locations for a number of depth and subdural electrodes. As shown, the electrodes include six right temporal depth (RTD) electrodes and six left temporal depth (LTD) electrodes located along the anterior-posterior plane in the hippocampi. FIG. 4A also includes four right orbitofrontal (ROF), four left orbitofrontal (LOF), four right subtemporal (RST) and four left subtemporal (LST) subdural electrodes located beneath the orbitofrontal and subtemporal cortical surfaces. FIG. 4B illustrates the placement of and use of a subdural electrode array as well as a strip of electrodes on the inferior right temporal lobe.

In accordance with an alternative embodiment of the present invention, magneto-electroencephalography (MEG) may be employed to record the magnetic fields produced by the brain. With MEG, an array of sensors called super-conducting quantum interference devices (SQUIDs) are used to detect and record the magnetic fields associated with the brain's internal current sources.

In yet another alternative embodiment, micro-electrodes may be implanted into the brain to measure the field potentials associated with one or just a few neurons. It will be understood that the use of micro-electrodes might be advantageous in very select applications, where, for example, it might be necessary to define with a high degree of accuracy the location of the epileptogenic focus prior to a surgical procedure.

Step 305 involves pre-processing the signals associated with each channel. Pre-processing includes, for example, signal amplification, filtering and digitization. In an embodiment, filters, including a high pass filter with 0.1 to 1 Hz cutoff and a low pass filter with 70-200 Hz cutoff, are employed. Depending on the application and/or the recording environment, other filters may be employed. For instance, if the signals are being recorded in the vicinity of power lines or any electrical fixtures or appliances operating on a 60 Hz cycle, a 60 Hz notch filter or time varying digital filters may be employed. Pre-processing results in the generation of a digital time series for each channel. Each digital time series signal is then sampled per step 307.

Step 309 involves generating phase-space portraits, and in particular, p-dimensional phase-space portraits for each channel, where p represents the number of dimensions necessary to properly embed a brain state. It will be understood that the actual value of p may depend upon which dynamical metrics are being relied upon to quantify the non-linear dynamical properties of each channel. For example, with regard to STL_{MAX}, it has been determined that p equal to seven is adequate to capture the dynamic characteristics of the spatio-temporal response associated with a given channel. However, in the case of ApEn and PM-ApEn, a value of p equal to 2-3 may be more appropriate.

In an embodiment of the present invention, the p-dimensional phase-space portraits are generated as follows. First, the digital signals associated with each channel are sampled over non-overlapping or overlapping sequential time segments, referred to herein as “epochs.” Each epoch may range in duration from approximately 5 seconds to approximately 24 seconds, depending upon signal characteristics such as frequency content, amplitude, dynamic properties (e.g., chaoticity or complexity) and stationarity. Generally, epoch length increases as stationarity increases.

The samples associated with each signal, taken during a given epoch, are then used to construct a phase-space portrait for the corresponding channel. In an embodiment of the present invention, the phase-space portraits are constructed using a method called “The Method of Delays.” The Method of Delays is well known in the art. A detailed discussion of this method with respect to analyzing dynamic, nonlinear systems can be found, for example, in Iasemidis et al., “Phase Space Topography of the Electrocorticogram and the Lyapunov Exponent in Partial Seizures”, Brain Topography, vol. 2, pp. 187-201 (1990). In general, a phase-space portrait is constructed using the Method of Delays by independently treating each unique sequence of p consecutive sample values, separated by a time delay, as a point to be plotted in the p-dimensional phase space. In an exemplary implementation of the present invention, p equals 4 samples (20 milliseconds).

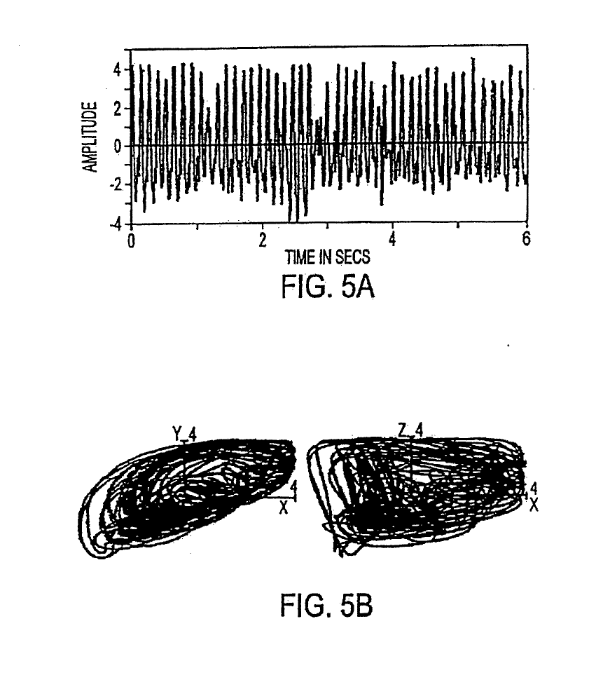

FIG. 5A shows a 6 second epoch associated with an exemplary EEG signal at the onset of a seizure that originated in the left temporal cortex. FIG. 5B illustrates, from different perspectives, the corresponding phase-space portrait, projected in three dimensions, for the exemplary EEG signal of FIG. 5A. The object appearing in the phase-space portrait of FIG. 5B is called an “attractor.” The attractor represents the region within the phase-space in which the states of the system evolve and remain confined thereto until the structure of the system changes.

Step 311 involves quantifying the dynamical properties of the spatio-temporal responses of each channel, as embodied by an attractor(s) associated with each channel, in accordance with each of the multiple dynamical metrics: STL_{MAX} (rate of divergence), Ω_{MAX} (rate of change in angular frequency), ApEn (approximate entropy or regularity statistic), PM-ApEn (pattern-match approximate entropy), h_{μ} (Pesin's Identity) and d_{L} (Lyapunov Dimension). Again, it will be understood that the list of dynamical metrics illustrated in FIG. 3 are exemplary, and not intended to be exhaustive.

As explained above, each dynamical profile represents the dynamical characteristics of a corresponding channel based on one of the multiple dynamical metrics, for example, STL_{MAX}, Ω_{MAX}, ApEn, PM-ApEn, h_{μ} and d_{L}. Calculating the dynamical values associated with each of these exemplary dynamical metrics is now described herein below.

L_{MAX} refers to the largest Lyapunov exponent. It is defined as the average of the local Lyapunov exponents Lᵢⱼ in the phase space where:

$${L}_{MAX} = \frac{1}{{N}_{a}} \cdot {\sum }_{a} {L}_{ij} ,$$

where Nₐ is the total number of the local Lyapunov exponents estimated from the evolution of adjacent vectors in the phase space. Lᵢⱼ is defined by the follow equation:

$${L}_{ij} = \frac{1}{Δ} t \cdot {log}_{2} \frac{\vert X ( {t}_{1} + Δ t ) - X ( {t}_{j} + Δ t ) \vert}{\vert X ( {t}_{i} ) - X ( {t}_{j} ) \vert} ,$$

where Xᵢ=X(tᵢ) and Xⱼ=X(tⱼ), and where Δt is the evolution time allowed for the vector difference δ₀(Xᵢⱼ)=|X(tᵢ)−X(tⱼ)| to evolve to the new difference δₖ(Xᵢⱼ)=|X(tᵢ+Δt)−X(tⱼ+Δt)|, and where Δt=k*dt with dt being the sampling period of the data u(t). If Δt is given in seconds, L_{MAX} is in bits/sec. STL_{MAX} is the short-term Lyapunov exponent. A method for computing STL_{MAX} is described in lasemidis et al., “Phase Space Topography of the Electrocorticogram and the Lyapunov Exponent in Partial Seizures,” Brain Topography, vol. 2, no. 3, pp. 187-201 (1990).

The difference in phase (ΔΦ) in the phase space is defined as the average of the local differences ΔΦᵢ as given by the following equation:

$$ΔΦ = \frac{1}{{N}_{a}} \cdot {\sum }_{i = 1}^{{N}_{a}} {ΔΦ}_{i} ,$$

where Nₐ is the total number of phase differences estimated from the evolution of X(tᵢ) to X(tᵢ+Δt) in the phase space, and where ΔΦᵢ is given by the following equation.

$$ΔΦ = \vert arc cos ( \frac{X ( {t}_{i} ) \cdot X ( {t}_{i} + Δ t )}{‖ X ( {t}_{i} ) ‖ \cdot ‖ X ( {t}_{i} + Δ r ‖} ) \vert .$$

The rate of angular frequency change (Ω_{MAX}) is then given by the following equation.

$${Ω}_{MAX} = \frac{1}{Δ t} ΔΦ$$

If Δt is given in seconds, then Ω_{MAX} is in radians/second. Further information regarding the computation of Ω_{MAX} may be found, for example, in lasemidis et al., “Prediction of Human Epileptic Seizures Based on Optimization and Phase Changes of Brain Electrical Activity,” Optimization Methods and Software, 18(1):81-104 (2003).

Entropy has been shown to be a critical “summary” statistic in nonlinear dynamical system analysis and chaos. Approximate Entropy (ApEn) measures the regularity of an observed time series data signal. See Pincus, “Approximate Entropy as a Measure of System Complexity,” Proceedings of the National Academy of Science of the United States of America, vol. 88 pp. 2297-2301 (1991). It has been widely used in distinguishing normal and abnormal medical time series data (e.g., heart rate and electrocardiogram data). ApEn may be calculated in accordance with the following process:
(a) given a time series data signal U=(u₁, u₂, . . . , uₙ}, where each entry is equally spaced in time;
(b) fix an integer l; where xᵢ=(uᵢ, uᵢ₊₁, . . . , uᵢ₊ₗ₋₁};
(c) form a sequence of vectors x₁, x₂, . . . , xₙ₋ₗ₊₁ in R¹;
(d) for a given positive real number r, use the sequence x₁, x₂, . . . , xₙ₋ₗ₊₁ to construct, for each i, 1≦i≦n−l+1, where, the following:

$${C}_{i}^{l} ( r ) = \frac{number of {x}_{j} ’ s such that d ( {x}_{i} , {x}_{j} ) \leq r}{n - l + 1} , \\ where$$

$$d ( {x}_{i} , {x}_{j} ) = {max}_{0 \leq k \leq l - 1} \vert {u}_{i + k} - {u}_{j + k} \vert , \\ i . e . ,$$

d(xᵢ, xⱼ) represents the maximum distance between vectors xᵢ and xⱼ in their respective scalar components;
(e) define the following:

$${Φ}^{l} ( r ) = {\sum }_{i = 1}^{n - l + 1} ln {C}_{i}^{l} ( r ) / ( n - l + 1 )$$
(f) then Approximate Entropy ApEn is defined in accordance with the following equation:
   −ApEn=Φ^{l}(r)−Φ^{l+1}(r)
(g) where it is noted that:

$$- ApEn = {Φ}^{l} ( r ) - {Φ}^{l} ( r ) \approx \frac{1}{n - l} {\sum }_{i = 1}^{n - l} ln \frac{{C}_{i}^{l + 1} ( r )}{{C}_{i}^{l} ( r )} , \\ and where$$

$$\frac{{C}_{i}^{l + 1} ( r )}{{C}_{i}^{l} ( r )} = \frac{Pr ( \vert {u}_{j + k} - {u}_{i + k} \vert \leq r , k = 0 , 1 , … , l )}{Pr ( \vert {u}_{j + k} - {u}_{i + k} \vert \leq r , k = 0 , 1 , … , l - 1 )} , \\ which in turn equals$$

$$Pr ( \vert {u}_{j + 1} - {u}_{i + l} \vert \leq r \vert {u}_{j + k} - {u}_{i + k} \vert \leq r , k = 0 \sim l - 1 )$$

Given the algorithm described above for calculating ApEn, it is noted that the calculation of ApEn is based on the following conditional probability.

Pr(next point value matched|the previous l points all value matched)

Because value match criterion is very sensitive to the matching critical value r, ApEn gives inconsistent results for different choices of parameters l and r. Accordingly, PM-ApEn can be derived as follows:
(a) given a time series data signal U=(u₁, u₂, . . . , uₙ}, where σᵤ is the sample standard deviation of U, and where, for a fixed integer l, a subsequence of U is defined as follows:
   xᵢ={uᵢ, uᵢ₊₁, . . . , uᵢ₊ₗ₋₁}, where
   1≦i≦n−1+l;
(b) for a given positive real number r (e.g., r=0.2σᵤ), xᵢ and xⱼ are considered pattern l-matched to each other if the following is true:
   |uᵢ−uⱼ|≦r;
   |uᵢ₊ₗ₋₁−uⱼ₊ₗ₋₁≦r; and
   sign(uᵢ₊ₖ−uᵢ₊ₖ₋₁)=sign(uⱼ₊ₖ−uⱼ₊ₖ₋₁), k1, . . . , l−1
(c) then, Pattern-Match ApEn is defined by an equation similar to the equation for ApEn, but change value match to pattern match, where:
   pᵢ=Pr(sign(uᵢ₊ₗ−uᵢ₊ₗ₋₁)=sign(uⱼ₊ₗ−uⱼ₊ₗ₋₁)|previous l points are pattern l-matched}
(d) the Pattern-Match ApEn can then be written as follows:

$$PM - ApEn = - \frac{1}{n - 1} {\sum }_{i = 1}^{m - 1} ln {\hat{p}}_{i}$$

where it should be noted that when the time series is more regular, the PM-ApEn should be smaller.

Lyapunov exponents may be used to define the metric entropy of EEG signals through Pesin's theorem. The Lyapunov spectrum (i.e., all Lyapunov exponents) and the metric entropy are related by Pesin's Identity, which may be defined as follows:

$${h}_{μ} = {\sum }_{i = 1}^{r} positive {λ}_{i} ,$$

where λᵢ represents the i^{th} Lyapunov exponent and r represents the maximum number of positive Lyapunov exponents.

The Lyapunov dimension is a characteristic related to the Lyapunov spectrum and the predictability of EEG signals. The state-space hypersurface of dimension m of the EEG signals expands at a rate governed by the sum of all Lyapunov exponents. Thus, Lyapunov Dimension may be defined in accordance with the following equation:

$${d}_{L} = k + \frac{{\sum }_{i = 1}^{k} {λ}_{i}}{\vert {λ}_{k + 1} \vert} ,$$

where k represents the largest number such that Σᵢ₌₁^{k}λᵢ>0, and the second term characterizes the fractional part of the dimension. It may be anticipated that, for an epileptic attractor, the Lyapunov dimension is equal to the information dimension.

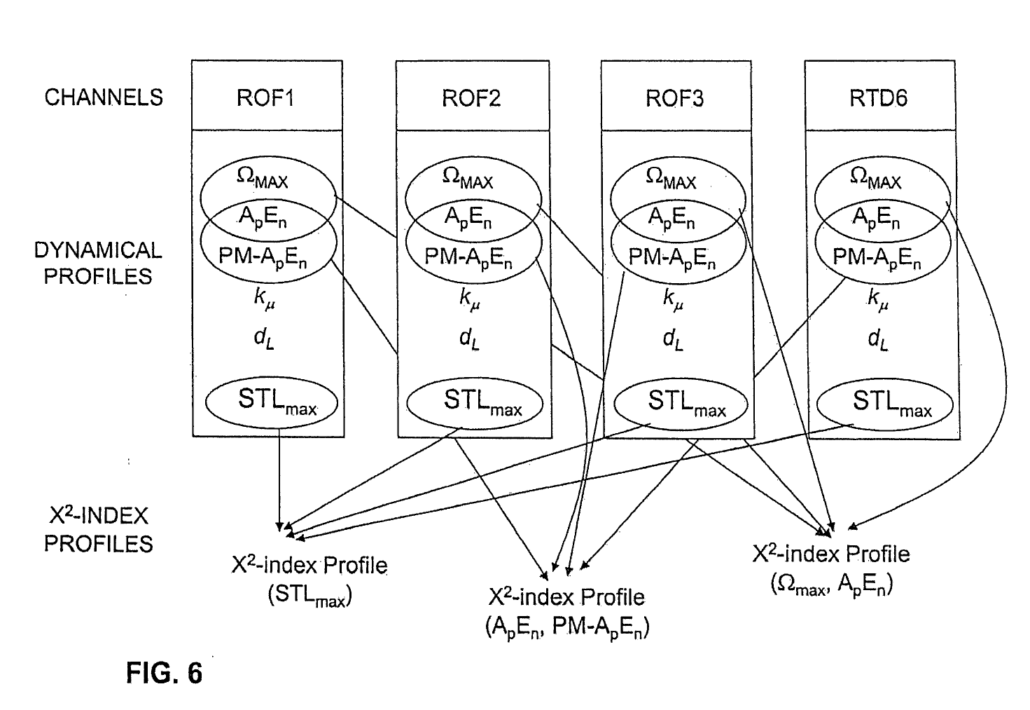

In accordance with an embodiment, step 311, as stated, further involves generating several X²-index profiles for at least one channel group, where each X²-index profile is based on corresponding dynamical profiles associated with one or a combination of dynamical metrics. This is illustrated in FIG. 6, where the algorithm is shown to have generated X²-index profiles for the exemplary channel group: ROF1-ROF2-ROF3-RTD6. It will be understood, however, the algorithm may generate additional X²-index profiles based on the dynamical profiles of other dynamical metrics or combinations thereof. Moreover, it will be understood that X²-index profiles may generated by the algorithm for additional channel groups.

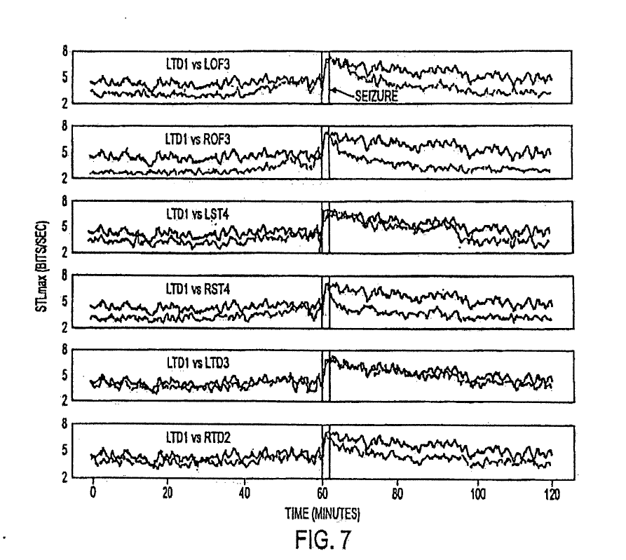

The nonparametric randomized block ANOVA test is well known in the art. Generally, the algorithm applies this test to generate the X²-index profiles by comparing the dynamical profiles associated with a corresponding one or more dynamical metrics. For example, the algorithm generates the X²-index profile relating to Ω_{MAX} and ApEn, as illustrated in FIG. 6, by comparing the dynamical profiles associated with Ω_{MAX} and ApEn for each channel of the channel group (e.g., the channel group ROF1-ROF2-ROF3-RTD6). If, at the end of the initialization period, Ω_{MAX} and ApEn are selected in accordance with method step 317, the X²-index profile thereafter generated by the algorithm based on the dynamical profiles associated with Ω_{MAX} and ApEn, for at least one channel group (e.g., the channel group ROF1-ROF2-ROF3-RTD6), is used to determine whether the spatio-temporal responses associated with the channels that make up the at least one channel group show signs of entrainment. For the purpose of the present invention, the term “entrain” refers to a correlation or convergence in amplitude and/or phase among the channels that make up the channel group. FIG. 7 illustrates, for example, a comparison of the dynamical profiles associated with the dynamical parameter STL_{MAX}, for each of a representative number of channel groups comprising two channels each.

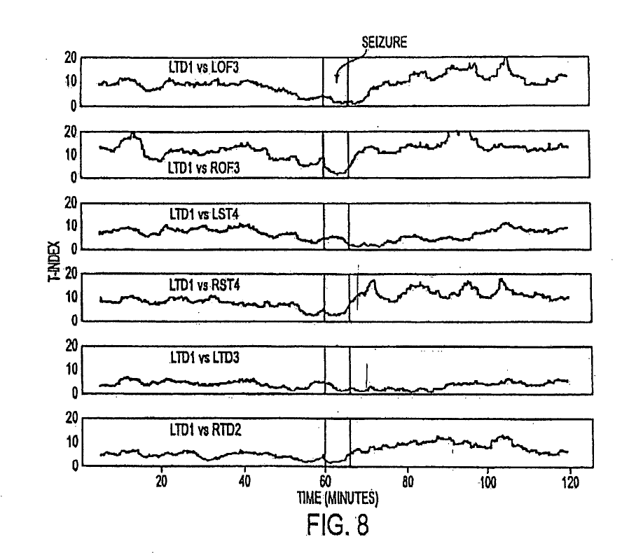

FIG. 8 illustrates statistical profiles, in this instance T-index profiles, for the six channel groups shown in FIG. 7. In FIG. 8, the statistical profiles are, as stated, T-index profiles, as they are generated by the algorithm based on a signal dynamical metric, STL_{MAX}, for channel groups comprising only two channels (i.e., channel pairs). From the T-index profiles illustrated in FIG. 8, it is evident that the STL_{MAX} profiles associated with each channel pair all progressively become entrained during the preictal stage (i.e., during the 0-60 minute time frame), while each channel pair becomes progressively disentrained during the postictal stage. However, the rate and degree at which the STL_{MAX} profiles become entrained and disentrained vary. In FIG. 8, the channel pair associated with the electrode LTD1 and electrode LTD3 demonstrates a relatively high level of entrainment (i.e., relatively low T-index values), more so than the other five channel pairs. The channel pair associated with the electrode LTD1 and electrode RTD2 also shows a relatively high level of entrainment, particularly during the preictal stage. Although FIG. 8 only shows T-index values 60 minutes prior to and 60 minutes following seizure onset, the preictal period typically begins approximately 15 minutes to as much as 2 hours prior to seizure onset. However, it is extremely important to note that signs of entrainment, such as reduced T-index values may be evident long before seizure onset. In fact, it is possible that some channels will exhibit signs of an impending seizure days before an actual seizure. Again, it is noted that while FIG. 8 illustrates the entrainment of STL_{MAX} dynamical profiles, by application of a T-statistic, the present invention may rely on dynamical profiles associated with multiple dynamical metrics, thus generating X²-index profiles, which may prove to be better indicators of impending seizures than STL_{MAX} alone.

Step 323 involves generating an ISW, SSPD and/or a TISP. The specific techniques employed to generate an ISW, SSPD and/or TISP, in accordance with this step, will now be described in greater detail. The first of these features to be described is the ISW feature. In accordance with the method of FIG. 3, an ISW is triggered when the X²-index values associated with the at least one channel group become highly disentrained (e.g., the X²-index value exceeds X²_{D}) followed by the X²-index values becoming highly entrained (e.g., the X²-index value falls below X²_{E}) for a statistically significant period of time. Further in accordance with an embodiment, the statistically significant period of time during which the X²-index value must remain below X²_{E} in order to trigger an ISW is typically set somewhere between 0 minutes and 1.5 hours. For example, an average X²-index value less than X²_{E} for a period of time equal to 15 minutes equates to a 99 percent confidence level that the issuance of an ISW is a valid warning. Of course, it will be understood that the threshold value X²_{E} and the duration which the X²-index must remain below that threshold value may be adaptively adjusted to increase or decrease ISW sensitivity and reduce the incidence of false warnings (i.e., false positives) for any given patient, or reduce the incidence of failed warnings (i.e., false negatives).

The ISW may be implemented in any number of ways. For example, the ISW may involve audible warnings or visual warnings or a combination of both visual and audible warnings. In fact, the ISW may involve nothing more than the setting or resetting of an internal software variable or flag, wherein the setting or resetting of the variable or flag triggers a dependent event, such as the automatic delivery of anti-seizure medication. Accordingly, the specific implementation of the ISW will depend on the specific clinical or non-clinical application for which the present invention is being employed.

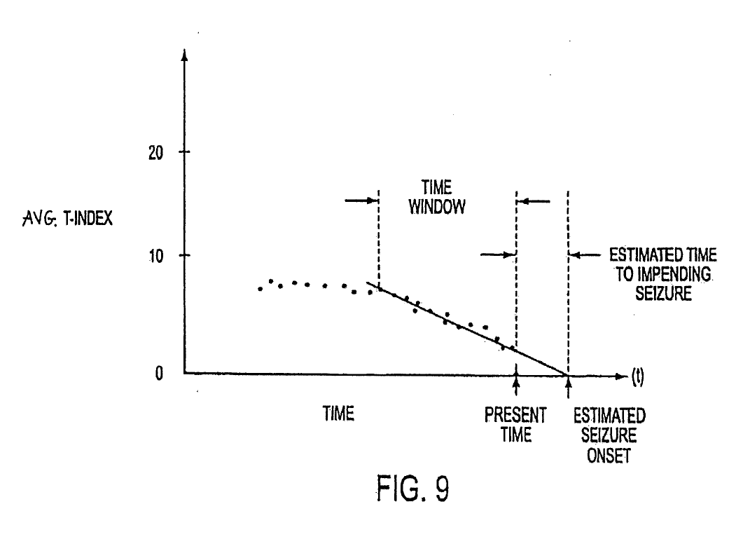

The next feature is the TISP feature. Once the algorithm generates an ISW, the rate of entrainment, that is, the rate at which the dynamical profiles associated with the selected one or combination of dynamical metrics, for the at least one channel group, continue to converge may be used to estimate the amount of time before seizure onset. In accordance with an embodiment of the present invention, this is accomplished by continuously deriving, for the at least one channel group, the slope of the corresponding X²-index profile over a “sliding” time window, as illustrated in FIG. 9. The point at which the slope intercepts the time (t) axis represents an estimated seizure onset time. Therefore, the difference between the present time and the estimated seizure onset time, along the time (t) axis, represents the TISP. The length of the “sliding” time window may, once again, vary. Initially, it may be set to a relatively small time interval (e.g., 15 minutes). Thereafter, it may be adaptively optimized for each individual patient.

The last of the three features is the SSPD feature. Over a period of several hours, if not several days prior to a seizure, or a first of a series of seizures, there is generally a gradual entrainment among certain critical sites. The concept of critical sites, critical channel pairs and critical channel groups is more fully set forth in U.S. Pat. No. 6,304,775 and co-pending U.S. patent application Ser. No. 10/648,354. The present invention exploits this to provide the SSPD feature. Specifically, the SSPD feature is, in accordance with an embodiment of the present invention, implemented in much the same way as the ISW feature, that is, by generating an X²-index profile, based on the selected one or combination of dynamical metrics, for each of the at least one channel groups, and by observing the levels of entrainment associated with those X²-index profiles. The X²-index profiles may be generated and observed over a period of several hours or days, rather than minutes.

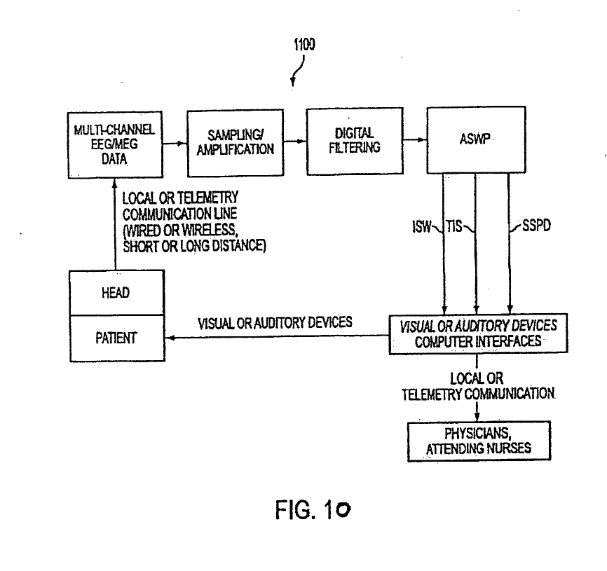

As one skilled in the art will readily appreciate, the aforementioned methods will be implemented as an integral component of a larger system. FIG. 10 is a block diagram of an exemplary system. This and other such exemplary systems are fully described in U.S. Pat. No. 6,304,775 and co-pending U.S. patent application Ser. No. 10/648,354. These systems may be employed for diagnostics, patient treatment, seizure intervention and, potentially, many other clinical and non-clinical applications.

The present invention has been described with reference to a number of exemplary embodiments. However, it will be apparent to those skilled in the art that it is possible to embody the invention in specific forms other than those described above without departing from the spirit of the invention. In fact, it will be readily apparent that the present invention may be employed for other medical (e.g., heart pacemakers, stroke diagnosis and prevention, dynamic brain disorders, etc.), non-medical, non-linear, multi-dimensional dynamic processes characterized by sudden phase transitions. Accordingly, the various embodiments described above are illustrative, and they should not be considered restrictive in any way. The scope of the invention is given by the appended claims, rather than the preceding description, and all variations and equivalents thereof that fall within the range of the claims are intended to be embraced therein.

What is claimed is:

1. A method of monitoring dynamical behavior of a multi-dimensional system comprising:
generating a multi-dimensional representation for each of a plurality of signals acquired from corresponding channels of the multi-dimensional system;
generating a plurality of dynamical profiles for each channel based on the corresponding multi-dimensional representations, wherein each dynamical profile reflects dynamical characteristics of the associated channel in accordance with one of a plurality of dynamical metrics;
calculating a plurality of first statistical measures for a group of a plurality of the channels, wherein each first statistical measure reflects a level of interaction among the channel group associated with at least one of the plurality of dynamical metrics;
calculating in an initialization period a second statistical measure for each of the first statistical measures, wherein the second statistical measure reflects a level of association of the first statistical measure with related occurrences being monitored;
selecting in the initialization period at least one of the plurality of dynamical metrics based on the second statistical measures calculated for each of the plurality of first statistical measures; and
identifying in the initialization period the first statistical measures corresponding to the selected dynamical metrics to characterize the dynamical behavior of the multi-dimensional system.

2. The method of claim 1, further comprising analyzing a set of the dynamical metrics each with a set of parameters identified by user input.

3. The method of claim 2, wherein the set of the dynamical metrics includes at least one of a maximum short-term Lyapunov exponent (STL_{MAX}), a rate of change in angular frequency (Ω_{MAX}), an approximate entropy (ApEn), a pattern-match approximate entropy (PM-ApEn), a Pesin's identity (h_{μ}), and a Lyapunov dimension (d_{L}).

4. The method of claim 1, further comprising implementing a statistical test identified by user input for calculating the first statistical measures.

5. The method of claim 4, wherein the statistical test includes a nonparametric Analysis of Variance (ANOVA) test.

6. The method of claim 1, wherein the selecting step comprises selecting at least one of the plurality of dynamical metrics corresponding to the most robust first statistical measure during the initialization period based on the second statistical measures.

7. The method of claim 6, wherein the selecting step comprises selecting at least one of the plurality of dynamical metrics corresponding to the first statistical measure having a desired level of sensitivity and specificity during the initialization period based on the second statistical measures.

8. The method of claim 1, wherein the identifying step comprises identifying in the initialization period the first statistical measures corresponding to the selected dynamical metrics to predict at least one occurrence.

9. The method of claim 8, further comprising monitoring the identified first statistical measures for a condition indicative of the at least one occurrence.

10. The method of claim 9, further comprising comparing the identified first statistical measures to at least one threshold value.

11. The method of claim 9, further comprising implementing at least one of a warning and an intervention in response to detecting the condition indicative of the at least one occurrence.

12. The method of claim 1, further comprising:
repeating the calculating, selecting, and identifying steps in the initialization period for additional channel groups; and
selecting a particular channel group based on a comparison of the second statistical measures for the respective channel groups.

13. A computer readable medium having stored therein a program, which when executed causes a processor to perform the following functions for monitoring dynamical behavior of a multi-dimensional system:
generate a multi-dimensional representation for each of a plurality of signals acquired from corresponding channels of the multi-dimensional system;
generate a plurality of dynamical profiles for each channel based on the corresponding multi-dimensional representations, wherein each dynamical profile reflects dynamical characteristics of the associated channel in accordance with one of a plurality of dynamical metrics;
calculate a plurality of first statistical measures for a group of a plurality of the channels, wherein each first statistical measure reflects a level of interaction among the channel group associated with at least one of the plurality of dynamical metrics;
calculate in an initialization period a second statistical measure for each of the first statistical measures, wherein the second statistical measure reflects a level of association of the first statistical measure with related occurrences being monitored;
select in the initialization period at least one of the plurality of dynamical metrics based on the second statistical measures calculated for each of the plurality of first statistical measures; and
identify in the initialization period the first statistical measures corresponding to the selected dynamical metrics to characterize the dynamical behavior of the multi-dimensional system.

14. The computer readable medium of claim 13, wherein the program further causes the processor to analyze a set of the dynamical metrics each with a set of parameters identified by user input.

15. The computer readable medium of claim 14, wherein the set of the dynamical metrics includes at least one of a maximum short-term Lyapunov exponent (STL_{MAX}), a rate of change in angular frequency (Ω_{MAX}), an approximate entropy (ApEn), a pattern-match approximate entropy (PM-ApEn), a Pesin's identity (h_{μ}), and a Lyapunov dimension (d_{L}).

16. The computer readable medium of claim 13, wherein the program further causes the processor to implement a statistical test identified by user input for calculating the first statistical measures.

17. The computer readable medium of claim 16, wherein the statistical test includes a nonparametric Analysis of Variance (ANOVA) test.

18. The computer readable medium of claim 13, wherein the program further causes the processor to select at least one of the plurality of dynamical metrics corresponding to the most robust first statistical measure during the initialization period based on the second statistical measures.

19. The computer readable medium of claim 18, wherein the program further causes the processor to select at least one of the plurality of dynamical metrics corresponding to the first statistical measure having a desired level of sensitivity and specificity during the initialization period based on the second statistical measures.

20. The computer readable medium of claim 13, wherein the program further causes the processor to identify in the initialization period the first statistical measures corresponding to the selected dynamical metrics to predict at least one occurrence.

21. The computer readable medium of claim 20, wherein the program further causes the processor to monitor the identified first statistical measures for a condition indicative of the at least one occurrence.

22. The computer readable medium of claim 21, wherein the program further causes the processor to compare the identified first statistical measures to at least one threshold value.

23. The computer readable medium of claim 21, wherein the program further causes the processor to implement at least one of a warning and an intervention in response to detecting the condition indicative of the at least one occurrence.

24. The computer readable medium of claim 13, wherein the program further causes the processor to perform the following functions:
repeat the calculating, selecting, and identifying steps in the initialization period for additional channel groups; and
select a particular channel group based on a comparison of the second statistical measures for the respective channel groups.

25. A system for monitoring dynamical behavior of a multi-dimensional system, comprising:
a processing device that executes the following steps:
generating a multi-dimensional representation for each of a plurality of signals acquired from corresponding channels of the multi-dimensional system;
generating a plurality of dynamical profiles for each channel based on the corresponding multi-dimensional representations, wherein each dynamical profile reflects dynamical characteristics of the associated channel in accordance with one of a plurality of dynamical metrics;
calculating a plurality of first statistical measures for a group of a plurality of the channels, wherein each first statistical measure reflects a level of interaction among the channel group associated with at least one of the plurality of dynamical metrics;
calculating in an initialization period a second statistical measure for each of the first statistical measures, wherein the second statistical measure reflects a level of association of the first statistical measure with related occurrences being monitored;
selecting in the initialization period at least one of the plurality of dynamical metrics based on the second statistical measures calculated for each of the first statistical measures; and
identifying in the initialization period the first statistical measures corresponding to the selected dynamical parameters to characterize the dynamical behavior of the multi-dimensional system.

26. The system of claim 25, further comprising a display device coupled to the processing device for visualization of the acquired signals, the dynamical profiles, and the first and second statistical measures.

27. The system of claim 25, further comprising a data acquisition device coupled to the processing device, wherein the data acquisition device acquires the plurality of signals from at least one sensor of the multi-dimensional system.

28. The system of claim 25, further comprising an end user device coupled to the processing device, wherein the processing device transmits occurrence information to the end user device based on the characterization of the dynamical behavior of the multi-dimensional system.

29. The system of claim 28, wherein the end user device initiates intervention in response to the transmitted occurrence information.
